(19) United States
(12) Patent Application Publication
(10) Pub. No.: US 2024/0389830 A1
(43) Pub. Date: Nov. 28, 2024

(54) ENDOSCOPE SYSTEM, ENDOSCOPE MOVEMENT CONTROL METHOD, AND RECORDING MEDIUM

(71) Applicant: OLYMPUS CORPORATION, Tokyo (JP)

(72) Inventors: Ryota SASAI, Tokyo (JP); Hiroyuki TAKAYAMA, Tokyo (JP)

(73) Assignee: OLYMPUS CORPORATION, Tokyo (JP)

(21) Appl. No.: 18/670,108

(22) Filed: May 21, 2024

Related U.S. Application Data
(60) Provisional application No. 63/468,906, filed on May 25, 2023.

(51) Int. Cl. A61B 1/00; A61B 1/05; A61B 34/30

(52) U.S. Cl. CPC A61B 1/00011; A61B 1/00096; A61B 1/05; A61B 34/30; A61B 2034/305

(57) ABSTRACT

An endoscope system includes an endoscope including an insertion portion, an imaging sensor disposed at a proximal end thereof so as to be rotatable about a longitudinal axis, and an optical element that tilts an optical axis, a robotic arm, and at least one processor. The processor transmits a first signal to the moving device, detects a first moving direction of an object, estimates a first amount of rotation of the optical element by using first two images before and after the transmission with respect to the imaging sensor based on the first movement direction, transmits a second signal to the robotic arm, detects a second moving direction of the object by using second two images before and after the transmission, and estimates a second amount of rotation between the robotic arm and the endoscope based on the second moving direction and the first amount of rotation.

### CROSS-REFERENCE TO RELATED APPLICATIONS

This application claims the benefit of U.S. Provisional Application No. 63/468,906, filed May 25, 2023, which is hereby incorporated by reference herein in its entirety.

### TECHNICAL FIELD

The present disclosure relates to an endoscope system, an endoscope movement control method, and a recording medium.

### BACKGROUND

An endoscope system in which an endoscope is moved by a moving device such as an electric holder has been conventionally known. In order to move the endoscope accurately, it is desirable that the coordinate system of the endoscope and the coordinate system of the moving device match each other.

For example, when an operator remotely operates an electric holder using a user interface, the operator inputs a desired moving direction of the endoscope into a user interface based on an endoscopic image. When the coordinate system of the endoscope and the coordinate system of the moving device match each other, the user can intuitively and accurately move the endoscope in a desired direction. On the other hand, when the coordinate system of the endoscope and the coordinate system of the moving device do not match each other, the actual moving direction of the endoscope is different from the moving direction input to the user interface, which makes it difficult for the user to intuitively and accurately move the endoscope in a desired direction.

In a robot system in which a camera system is attached to a movable arm, there is known a method for correcting the transformation between the coordinate system of a robot and the coordinate system of a camera system (see, for example, PTL 1). In PTL 1, an image of a target is captured by a camera system, and the transformation is determined from the position of the movable arm when the image is captured and the position of a feature point of the target in the image.

### SUMMARY

According to an aspect of the present disclosure, an endoscope system comprises: an insertion portion extending in a longitudinal axis direction;
an imaging sensor disposed at a proximal end of the insertion portion so as to be rotatable about a longitudinal axis; an optical element provided in the insertion portion to tilt an optical axis in a direction offset from the longitudinal axis direction; a robotic arm that moves and holds the endoscope such that the endoscope is rotatable about the longitudinal axis; and at least one processor comprising hardware, wherein the processor is configured to: transmit, to the robotic arm, a first signal for moving the endoscope in the longitudinal axis direction, detect a first moving direction of an object within first two images by using the first two images captured by the imaging sensor before and after the transmission of the first signal, estimate a first amount of rotation of the optical element around the longitudinal axis with respect to the imaging sensor based on the first movement direction, transmit, to the moving device, a second signal for moving the endoscope perpendicular to the longitudinal axis direction, detect a second moving direction of the object within second two images by using the second two images captured by the imaging sensor before and after the transmission of the second signal, and estimate a second amount of rotation about the longitudinal axis between the robotic arm and the endoscope based on the second moving direction and the first amount of rotation.

According to another aspect of the present disclosure, there is provided an endoscope movement control method for controlling a robotic arm for moving an endoscope, the endoscope comprising an insertion portion extending in a longitudinal axis direction, an imaging sensor disposed at a proximal end of the insertion portion so as to be rotatable about a longitudinal axis, and an optical element that is provided in the insertion portion to tilt an optical axis in a direction offset from the longitudinal axis direction, and the robotic arm holding the endoscope such that the endoscope is rotatable about the longitudinal axis, the endoscope movement control method comprising: moving the endoscope in the longitudinal axis direction by the robotic arm; detecting a first moving direction of an object in first two images by using the first two images captured by the imaging sensor before and after the movement in the longitudinal axis direction; estimating a first amount of rotation of the optical element about the longitudinal axis with respect to the imaging sensor based on the first moving direction; moving the endoscope in a direction perpendicular to the longitudinal axis direction by the robotic arm; detecting a second moving direction of the object in second two images using two images captured by the imaging sensor before and after the movement in the direction perpendicular to the longitudinal axis direction; and estimating a second amount of rotation about the longitudinal axis between the robotic arm and the endoscope based on the second moving direction and the first amount of rotation.

According to another aspect of the present disclosure, there is provided a non-transitory computer-readable recording medium in which an endoscope movement control program for controlling a robotic arm for moving an endoscope is stored, the endoscope comprising an insertion portion extending in a longitudinal axis direction, an imaging sensor disposed at a proximal end of the insertion portion so as to be rotatable about a longitudinal axis, and an optical element that is provided in the insertion portion to incline an optical axis in a direction offset from the longitudinal axis direction, the robotic arm holding the endoscope such that the endoscope is rotatable about the longitudinal axis, and the endoscope movement control program causing a computer to: move the endoscope in the longitudinal axis direction by the robotic arm; detect a first moving direction of an object in first two images by using two images captured by the imaging sensor before and after the movement in the longitudinal axis direction; estimate a first amount of rotation of the optical element about the longitudinal axis with respect to the imaging sensor based on the first moving direction; move the endoscope in a direction perpendicular to the longitudinal axis direction by the robotic arm; detect a second moving direction of the object in the second two images by using the second two images captured by the imaging sensor before and after the movement in the direction perpendicular to the longitudinal axis direction; and estimate a second amount of rotation about the longitudinal axis between the robotic arm and the endoscope based on the second moving direction and the first amount of rotation.

### BRIEF DESCRIPTION OF DRAWINGS

FIG. 1 is an overall configuration diagram of an endoscope system according to a first embodiment of the present disclosure.

FIG. 2 is a block diagram showing the overall configuration of the endoscope system in FIG. 1.

FIG. 3 is a diagram showing an example of a treatment tool area and other areas in an endoscopic image.

FIG. 4 is a partial longitudinal sectional view showing the endoscope and a trocar of the endoscope system of FIG. 1.

FIG. 5A is a flowchart illustrating an endoscope movement control method by the endoscope system of FIG. 1.

FIG. 5B is a flowchart following FIG. 5A.

FIG. 6 is a diagram showing an example of a movement vector calculated from an endoscopic image.

FIG. 7 is a diagram showing an example of a temporary vanishing point set in the endoscopic image.

FIG. 8 is a diagram showing the relation between a viewing angle and the vanishing point of the endoscope.

FIG. 9 is a diagram illustrating processing for determining a vanishing point from the temporary vanishing point set in FIG. 7.

FIG. 10 is a diagram illustrating processing for determining an amount of rotation θ from a movement vector acquired from endoscopic images before and after movement in a direction perpendicular to a longitudinal axis of the endoscope.

FIG. 11 is a diagram showing a first modification of the endoscope system shown in FIG. 1, and showing an example of a treatment tool area, a margin, and other areas in an endoscopic image.

FIG. 12 is a diagram illustrating a third modification of the endoscope system in FIG. 1, and illustrating processing for determining a vanishing point from a movement vector in FIG. 6.

FIG. 13 is a diagram showing a fifth modification of the endoscope system of FIG. 1, and showing a flowchart following FIG. 5A.

FIG. 14 is a diagram showing a sixth modification of the endoscope system of FIG. 1, and showing a flowchart replacing FIG. 5A.

FIG. 15 is a diagram illustrating an operation of a moving device according to a third signal in the flowchart of FIG. 14.

FIG. 16 is a diagram showing a seventh modification of the endoscope system of FIG. 1, and showing a flowchart following FIG. 5A.

FIG. 17 is a diagram showing an eighth modification of the endoscope system of FIG. 1, and showing a flowchart following FIG. 5A.

FIG. 18 is a partial longitudinal sectional view of an endoscope and a trocar that shows a ninth modification of the endoscope system of FIG. 1.

FIG. 19 is a flowchart illustrating registration processing of a registration point in the endoscope system of FIG. 18.

FIG. 20 is a diagram illustrating the distances between the distal end of the endoscope and a plurality of registration points and a pivot point in the endoscope system of FIG. 18.

FIG. 21 is a diagram showing a flowchart replacing FIG. 5A of the endoscope system of FIG. 18.

FIG. 22 is a diagram showing the relation of an angle of a curved joint of an endoscope, an angle of view, and a vanishing point in an endoscope system according to a second embodiment of the present disclosure.

FIG. 23 is a diagram illustrating the amount of rotation about the longitudinal axis of the endoscope in the endoscope system of FIG. 22.

FIG. 24 is a diagram showing a flowchart replacing FIG. 5A of the endoscope system of FIG. 22.

FIG. 25 is a diagram illustrating processing for determining the distance between the center of an image and a vanishing point and the amount of rotation in the endoscope system of FIG. 22.

### DESCRIPTION OF EMBODIMENTS

### First Embodiment

An endoscope system 1, an endoscope movement control device 5, a method, a program, and a recording medium according to a first embodiment of the present disclosure will be described with reference to the drawings.

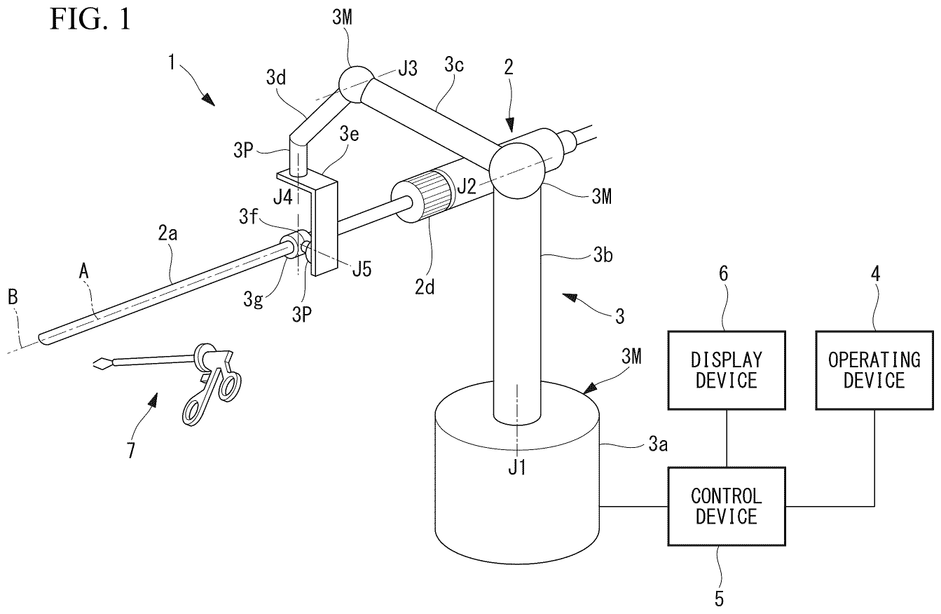

As shown in FIG. 1, the endoscope system 1 according to the present embodiment includes an endoscope 2, a moving device 3 for holding and moving the endoscope 2, an operating device 4 to be operated by a user, a control device (endoscope movement control device) 5 for controlling the moving device 3 based on an operation signal from the operating device 4, and a display device 6.

The endoscope 2 is a rigid endoscope including an elongated and rigid lens tube portion (insertion portion) 2a, an optical system (optical element) 2b disposed in the lens tube portion 2a, and a camera head 2c disposed at a base end of the lens tube portion 2a. Furthermore, the endoscope 2 is an oblique-viewing type endoscope 2 having a visual axis (optical axis) B which is tilted at a predetermined angle by an optical system 2b with respect to a longitudinal axis A extending along the center in the radial direction of the lens tube portion 2a.

The lens tube portion 2a is attached to the camera head 2c so as to be rotatable around the longitudinal axis A. An operation ring 2d is fixed to the lens tube portion 2a. An operator can rotate the lens tube portion 2a around the longitudinal axis A with respect to the camera head 2c by rotating the operation ring 2d around the longitudinal axis A while holding the camera head 2c. Alternatively, the operator can also rotate the lens tube portion 2a around the longitudinal axis A with respect to the camera head 2c by rotating the camera head 2c around the longitudinal axis A while holding the operation ring 2d.

An imaging element 2e for imaging light focused by the optical system 2b is fixed inside the camera head 2c. The imaging element 2e includes an imaging surface which is arranged perpendicularly to the longitudinal axis A such that the longitudinal axis A passes through the center thereof. The imaging element 2e is an image sensor such as a CCD (Charge Coupled Device) image sensor or a CMOS (Complementary Metal Oxide Semiconductor) image sensor.

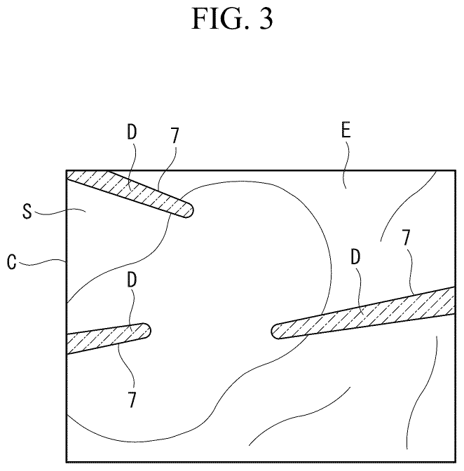

The endoscope 2 is inserted into a body together with one or more treatment tools 7, and an endoscopic image (image) C including the one or more treatment tools 7 (see FIG. 3) is captured by the imaging element 2e, and transmitted through the control device 5 to the display device 6. The display device 6 is any display such as a liquid crystal display or an organic EL display. The operator manipulates the treatment tools 7 while observing the endoscopic image C displayed on the display device 6.

The moving device 3 is a five-degree-of-freedom robot arm having a plurality of, for example, three driving joints (joints) 3M and two passive joints 3P. The moving device 3 includes, for example, a base 3a installed on a horizontal plane, and a first link 3b to be rotationally driven with respect to the base 3a around a vertical first axial line J1. Further, the moving device 3 includes a second link 3c to be rotationally driven with respect to the first link 3b around a horizontal second axial line J2, and a third link 3d to be rotationally driven with respect to the second link 3c around a horizontal third axial line J3. Further, the moving device 3 includes a fourth link 3e that is supported to be freely rotatable with respect to the third link 3d about a fourth axial line J4 extending along a plane that is orthogonal to the third axial line J3 and contains the first axial line J1, and a fifth link 3f which is supported to be rotatable with respect to the fourth link 3e around a fifth axial line J5 perpendicular to the fourth axial line J4.

The moving device 3 includes a holder 3g that is provided at the distal end of the fifth link 3f and holds the lens tube portion 2a of the endoscope 2 so as to make the lens tube portion 2a rotatable around the longitudinal axis A. The holder 3g holds the lens tube portion 2a such that the longitudinal axis A of the lens tube portion 2a passes through the intersection point between the fourth axial line J4 and the fifth axial line J5 and is perpendicular to the fifth axial line J5. The moving device 3 has five-degree-of-freedom. However, since the holder 3g holds the lens tube portion 2a so as to make the lens tube portion 2a rotatable around the longitudinal axis A, the moving device 3 has six-degree-of-freedom as a whole, so that the endoscope 2 can be arranged in a desired posture and at a desired position.

The endoscope 2 is moved by an interlocking operation of the three driving joints 3M and the two passive joints 3P of the moving device 3, whereby the position and posture of the endoscope 2 are three-dimensionally changed.

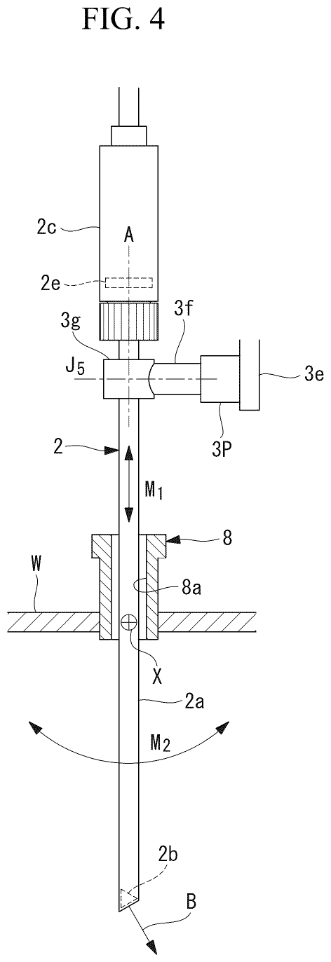

When using the endoscope 2, as shown in FIG. 4, the lens tube portion 2a of the endoscope 2 is inserted into a through-hole 8a of a trocar 8 that has been attached to an abdominal wall W of a patient by penetrating the trocar 8 through the abdominal wall W, thereby observing the inside of the patient's abdominal cavity. The trocar 8 is swingable about a pivot point X near the abdominal wall W, and the inner diameter of the through-hole 8a is set to be slightly larger than the outer diameter of the lens tube portion 2a. The gap between the through-hole 8a and the lens tube portion 2a is hermetically sealed by a sealing member made of an elastic material (not shown).

The three-dimensional position of the distal end of the third link 3d is uniquely determined by driving the three driving joints 3M. The moving direction of the endoscope 2 is restricted by the trocar 8. Therefore, when the distal end position of the third link 3d is changed, the endoscope 2 swings the trocar 8 around the pivot point X or move the lens tube portion 2a in the longitudinal axis direction A inside the through-hole 8a of the trocar 8, and passively rotates the two passive joints 3P so as to follow the above movement. The rotation of the lens tube portion 2a around the longitudinal axis A relative to the holder 3g is maintained in a state manually adjusted by the operator.

Each of the three driving joints 3M of the moving device 3 is equipped with an angle sensor 3h for detecting the rotation angle thereof. The angle sensor 3h is, for example, an encoder, a potentiometer, or a Hall sensor provided at each driving joint 3M.

The operating device 4 has a user interface 4a including input devices such as keys, a joystick, buttons, and a touch panel. The operator can input an instruction for moving the endoscope 2 to the operating device 4 by operating the user interface 4a. The operating device 4 transmits an operating signal based on an operation of the user interface 4a to the control device 5.

Further, the user interface 4a can accept a trigger input from the operator. As described later, the trigger is used for executing processing of estimating the amounts of rotation amounts β and θ of the endoscope 2.

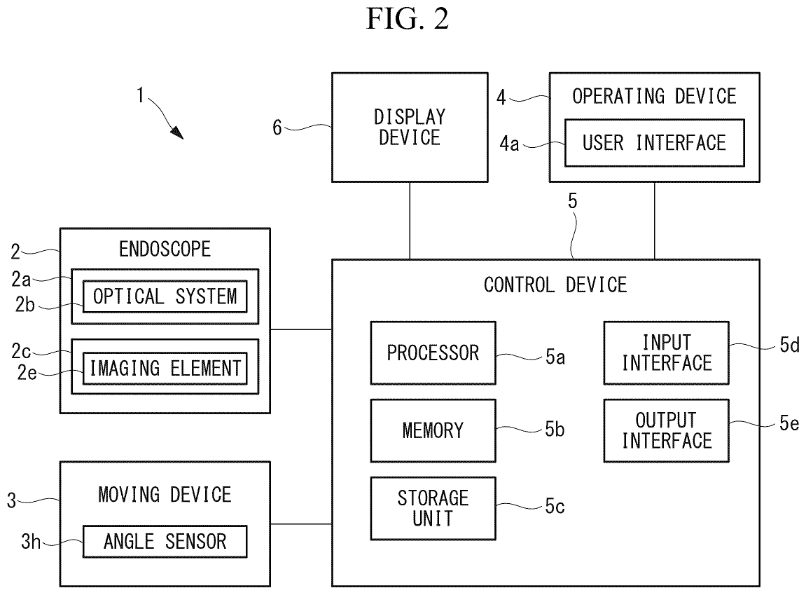

As shown in FIG. 2, the control device 5 includes at least one processor 5a, a memory 5b, a storage unit 5c, an input interface 5d, and an output interface 5e.

The control device 5 is connected to the surrounding endoscope 2, moving device 3 (robotic arm), operating device 4, and display device 6 via the input interface 5d and the output interface 5e, and transmits and receives an endoscopic image C, information on the rotation angle of the driving joints 3M, signals, etc. via the interfaces 5d and 5e.

The memory 5b is, for example, a semiconductor memory including ROM (read-only memory) or RAM (random access memory) area.

The storage unit 5c is a computer-readable non-transitory recording medium, and is, for example, a nonvolatile recording medium including a hard disk or a semiconductor memory such as a flash memory.

The processor 5a controls the moving device 3 based on the operation signal from the operating device 4, thereby moving the endoscope 2 according to an instruction input to the user interface 4a by the operator.

Here, the operator can rotate the endoscope 2 around the longitudinal axis A according to two ways by operating the endoscope 2 attached to the holder 3g. First, the lens tube portion 2a is rotated around the longitudinal axis A with respect to the camera head 2c by manipulating the operation ring 2d, which makes it possible to change the relative angle (first amount of rotation) β between the camera head 2c and the lens tube portion 2a. Second, the lens tube portion 2a is rotated around the longitudinal axis A with respect to the holder 3g, which makes it possible to change the angle (second amount of rotation) θ representing the direction of the visual axis B of the lens tube portion 2a with respect to the holder 3g.

In order for the operator to attach the endoscope 2 to the moving device 3 and intuitively move the endoscope 2 vertically and horizontally while watching the endoscopic image C displayed on the display device 6, it is necessary that the amounts of rotation β and θ between the moving device 3 and the endoscope 2 have been grasped.

In the endoscope system 1 according to the present embodiment, the processor 5a estimates the amounts of rotation β and θ of the endoscope 2 by executing a program (endoscope movement control program) which is recorded in the storage unit 5c and read out to the memory 5b.

Hereinafter, an endoscope movement control method by the endoscope system 1 according to the present embodiment will be described with reference to the drawings.

The processing of estimating the amounts of rotation β and θ of the endoscope 2 is started by the operator inputting a trigger to the user interface 4a.

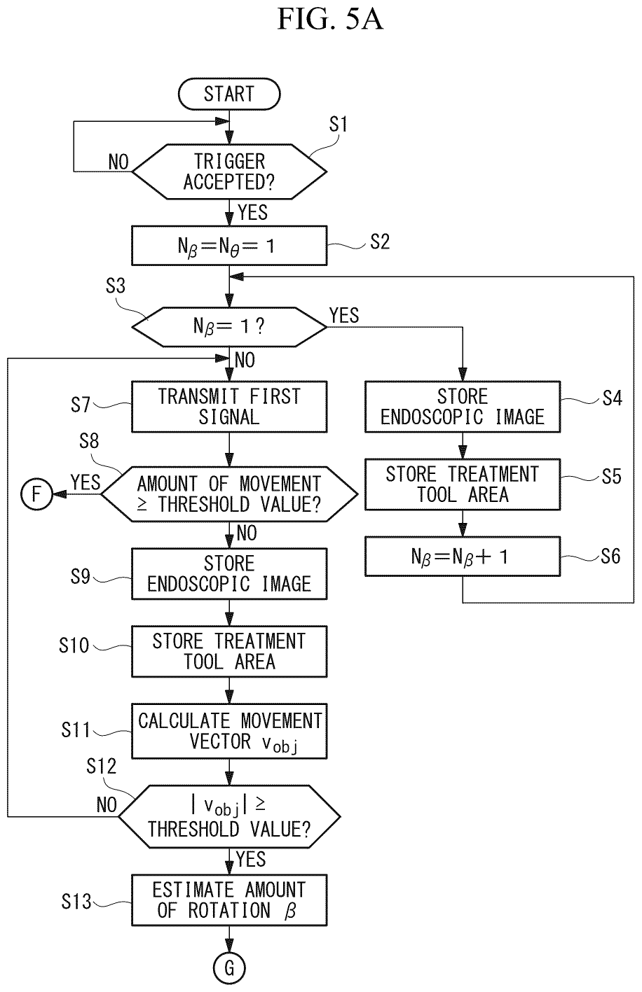

As shown in FIG. 5A, the processor 5a first determines whether a trigger has been received (step S1), and if accepted, the processor 5a initializes the numbers of loops NB and N_{θ}, respectively (step S2). Next, the processor 5a determines whether N_{β}=1 is satisfied (step S3), and if N_{β}=1 is satisfied, the processor 5a stores a first endoscopic image C acquired by the endoscope 2 at that time into the memory 5b (step S4). Further, the processor 5a recognizes treatment tools (moving objects) 7 in the first endoscopic image C by using a known method such as image recognition using artificial intelligence, and stores areas of the treatment tools 7 as treatment tool areas D into the memory 5b (step S5). Then, the processor 5a increments the number of loops N_{β} (step S6), and repeats the processing from step S3.

If the processor 5a determines in step S3 that N_{β}≠1 is satisfied, the processor 5a transmits a first signal for activating the moving device 3 so as to move the endoscope 2 in one direction along the longitudinal axis A (Step S7). It is determined whether the amount of movement of the endoscope 2 caused by the moving device 3 being activated with the first signal is equal to or more than a predetermined threshold value (step S8). If it is equal to or more than the threshold value, the processing is terminated.

When determining that the amount of movement of the endoscope 2 is smaller than the threshold value, the processor 5a stores a second endoscopic image C acquired by the endoscope 2 at that time into the memory 5b (step S9). Further, the processor 5a recognizes the treatment tools (moving objects) 7 in the second endoscopic image C, and stores the areas of the treatment tools 7 as treatment tool areas D into the memory 5b (step S10).

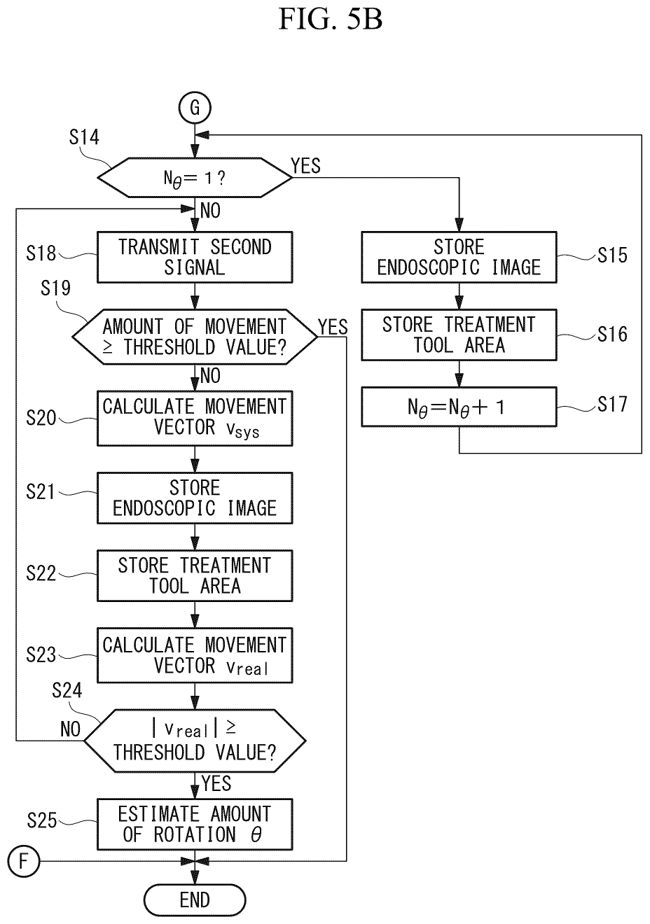

A first moving direction M₁ of an object S in the endoscopic image C is detected using the first endoscopic image C and the second endoscopic image C (step S11). Specifically, the processor 5a uses a known method such as optical flow to estimate a movement vector v_{obj} of the object S in another area E obtained by excluding the treatment tool areas D from the two endoscopic images C captured before and after the endoscope 2 is moved by the moving device 3. As shown in FIG. 6, the movement vector v_{obj} is a two-dimensional vector that indicates the moving direction of each of a plurality of different feature points in the other area E within the endoscopic image C.

Next, the processor 5a determines whether the magnitude of the estimated movement vector v_{obj} is equal to or more than a threshold value (step S12). The determination of the magnitude of the movement vector v_{obj} is performed using the magnitude of any of estimated movement vectors v_{obj} or the average value of the magnitudes of the estimated movement vectors v_{obj}.

When the magnitude of the movement vector v_{obj} is smaller than the threshold value, the processor 5a repeats the processing from step S7. When the magnitude of the movement vector v_{obj} is equal to or more than the threshold value, the processor 5a estimates the amount of rotation β of the endoscope 2 from the estimated movement vector v_{obj} (step S13).

The estimation of the amount of rotation β of the endoscope 2 is performed as follows.

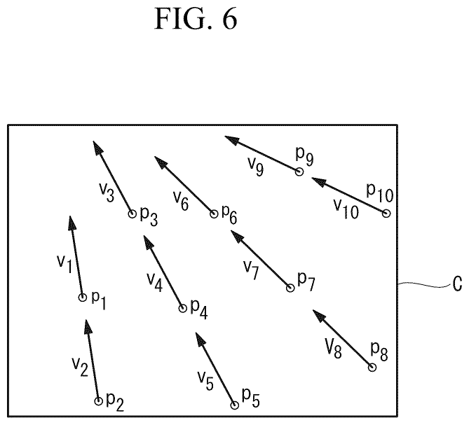

First, the processor 5a calculates the intersection point (hereinafter also referred to as a vanishing point) of straight lines along the directions of a plurality of movement vectors v_{obj} estimated for a plurality of feature points on the endoscopic image C. Specifically, as shown in FIG. 7, the processor 5a arranges a plurality of temporary vanishing points Q₁ to Q₁₂ at equal intervals on a circumference of a radius L from the center O (hereinafter referred to as an image center) of the endoscopic image C, and stores the respective positions of the temporary vanishing points Q₁ to Q₁₂. FIG. 7 shows an example in which the vanishing points Q₁ to Q₁₂ are arranged at intervals of 30°.

The radius L is the distance from the image center O to the vanishing point, and can be calculated using Formula (1).

$$[ Formula 1 ]$$

$$L = ( \frac{H \cdot tan ( α )}{2 \cdot tan ( ψ / 2 )} ) \qquad ( 1 )$$

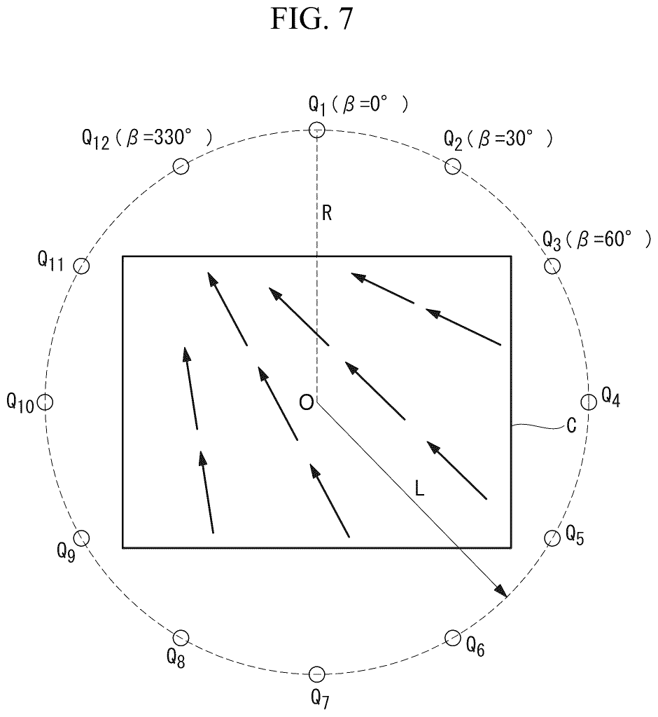

Here, as shown in FIG. 8, H represents the height (pixel) of the endoscopic image C, α represents the angle of the visual axis B with respect to the longitudinal axis A of the endoscope 2, and φ represents the field of view of the endoscope 2. For example, the angle α may be 30 degrees.

The processor 5a uses, as a reference line R, a straight line which connects the position of a vanishing point Q₁ for the amount of rotation β=0° and the image center O, and sets the temporary vanishing points Q₁ to Q₁₂, for example, at intersection points between the circle of the radius L and straight lines obtained by rotating the reference line R by every 30° around the image center O. As a result, for example, when the temporary vanishing point Q₂ at a position where rotation of 30° is performed with respect to the reference line R is selected as the vanishing point, the amount of rotation β of the endoscope 2 can be estimated to be β=30°.

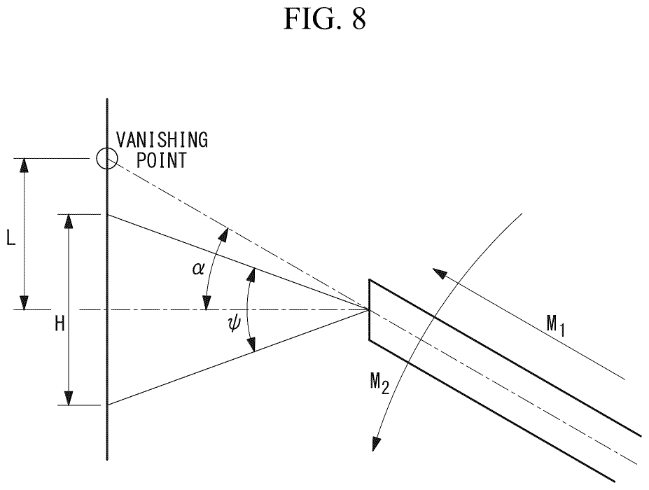

The processor 5a calculates, for each temporary vanishing point Qⱼ, the inner product of each movement vector vᵢ and a vector (Qⱼ−pᵢ) connecting the origin pᵢ of each movement vector vᵢ and the temporary vanishing point Qⱼ. FIG. 9 illustrates a vector (Q₂-p₆) connecting the temporary vanishing point Q₂ and the origin p₆ of the vector v₆ and a vector (Q₁₂-p₆) connecting the temporary vanishing point Q₁₂ and the origin p₆ of the vector v₆. Then, for example, as shown in Formula (2), the processor calculates the sum of inner products for all movement vectors vᵢ as an evaluation function Uⱼ, and selects, as the vanishing point, a temporary vanishing point Qⱼ where the evaluation function Uⱼ is minimized.

$$[ Formula 2 ]$$

$$\begin{matrix}{U}_{j} = {\sum }_{i = 1}^{N} ( {Q}_{j} - {p}_{i} ) \cdot {v}_{i} & ( 2 )\end{matrix}$$

In the example shown in FIG. 9, the temporary vanishing point Q₁₂ is selected as the vanishing point.

As a result, the processor 5a estimates the rotation angle corresponding to the selected vanishing point, 330° in the example shown in FIG. 9, as the amount of rotation β of the endoscope 2.

Next, as shown in FIG. 5B, the processor 5a determines whether the number of loops N_{θ} satisfies N_{θ}=1 (step S14). In case of N_{θ}=1, the third endoscopic image C captured by the endoscope 2 at that time is stored in the memory 5b (step S15). Further, the processor 5a stores the areas of the treatment tools (moving objects) 7 in the third endoscopic image C as the treatment tool areas D in the memory 5b in the same manner as in step S5 (step S16). Then, the processor 5a increments the number of loops Ne (step S17), and repeats the processing from step S14.

In step S14, when determining that Nθ≠1 is satisfied, the processor 5a transmits a second signal for causing the moving device 3 to operate so as to move the endoscope 2 in one direction perpendicular to the longitudinal axis A (step S18). The processor 5a determines whether the amount of movement of the endoscope 2 caused by the moving device 3 being activated with the second signal is equal to or more than a predetermined threshold value (step S19), and when it is equal to or more than the threshold value, the processor 5a terminates the rotation amount estimation processing.

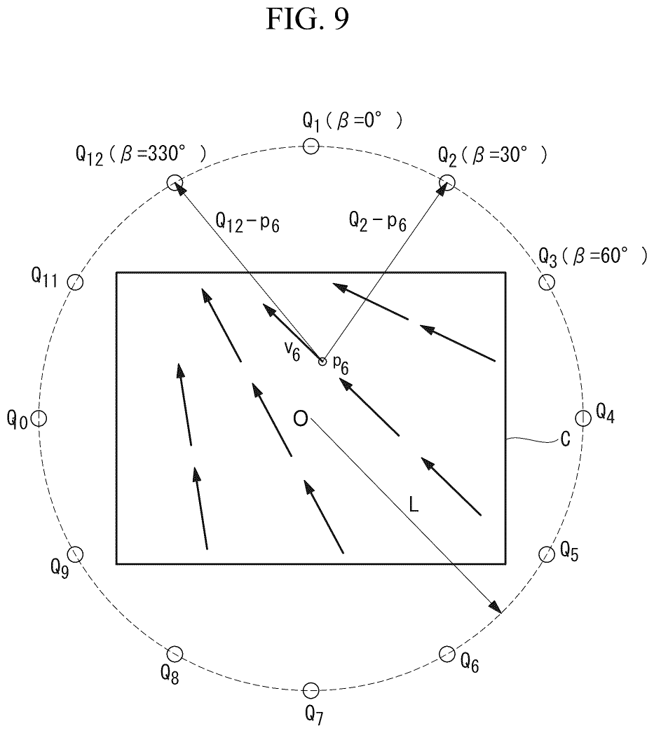

When the movement amount of the endoscope 2 is smaller than the threshold value, the processor 5a uses the amount of rotation β estimated in step S13 to calculate a movement vector v_{sys} of the object S in the endoscopic image C, which is assumed to be caused by transmission of the second signal when the amount of rotation θ is equal to a predetermined value, for example, 0° (step S20). FIG. 10 illustrates the movement vector v_{sys} in case of (β, θ)=(−90°, 0°).

Then, the processor 5a stores, into the memory 5b, the fourth endoscopic image C captured after the endoscope 2 is moved with the second signal (step S21). Further, the processor 5a recognizes the treatment tools (moving objects) 7 in the fourth endoscopic image C, and stores the areas of the treatment tools 7 as the treatment tool areas D into the memory 5b (step S22).

The second moving direction M₂ of the object S in the endoscopic image C is detected using the third endoscopic image C and the fourth endoscopic image C (step S23). Specifically, the processor 5a uses a known method such as optical flow to estimate a movement vector vᵣₑₐₗ of the object S in another area E obtained by excluding the treatment tool areas D from the two endoscopic images C captured before and after the endoscope 2 is moved by the moving device 3.

Next, the processor 5a determines whether the magnitude of the estimated movement vector vᵣₑₐₗ is equal to or more than a threshold value (step S24). The determination of the magnitude of the movement vector vᵣₑₐₗ is performed using the magnitude of any of estimated movement vectors vᵣₑₐₗ or the average value of the magnitudes of the estimated movement vectors vᵣₑₐₗ.

When the magnitude of the movement vector vᵣₑₐₗ is smaller than the threshold value, the processor 5a repeats the processing from step S18. When the magnitude of the movement vector vᵣₑₐₗ is equal to or more than the threshold value, the processor 5a estimates the angle between the assumed movement vector v_{sys} and the estimated movement vector vᵣₑₐₗ, which is 180° in the example shown in FIG. 10, as the amount of rotation θ of the endoscope 2 (step S25), and terminates the processing.

When the amounts of rotation β and θ of the endoscope 2 are estimated, the processor 5a uses the estimated amounts of rotation β and θ to correct a holder coordinate system Σr so that the holder coordinate system Σr and an endoscope coordinate system e match each other. The endoscope coordinate system Ce is a coordinate system fixed for the imaging element 2e, and the holder coordinate system Σr is a coordinate system fixed for the distal end of the holder 3g.

In one example, the endoscope coordinate system Ce is a rectangular coordinate system having Xe-axis, Ye-axis, and Ze-axis which are orthogonal to one another, and the holder coordinate system Σr is a rectangular coordinate system having Xr-axis, Yr-axis, and Zr-axis which are orthogonal to one another. The Xe-axis and the Xr-axis match the longitudinal axis A, the Ye-axis and the Yr-axis are parallel to the horizontal direction (left-right direction) of the endoscopic image C, and the Ze-axis and the Zr-axis are parallel to the vertical direction (up-and-down direction) of the endoscopic image C. The holder coordinate system Σr and the endoscope coordinate system Σe matching each other means that the directions of the Xe-axis and Xr-axis match each other, the directions of the Ye-axis and Yr-axis match each other, and the directions of the Ze-axis and Zr-axis match each other.

However, when the endoscope 2 is rotated by the amounts of rotation β and θ around the longitudinal axis A, a shift occurs between the endoscope coordinate system Ce and the holder coordinate system Σr. When the endoscope coordinate system Ce does not match the holder coordinate system Σr as described above, it is difficult for the operator who is observing the endoscopic image C displayed on the display device 6 to intuitively and accurately move the endoscope 2 in a desired direction by operating the user interface 4a.

The processor 5a makes the holder coordinate system Σr match the endoscope coordinate system Ce by correcting the holder coordinate system Σr based on the amounts of rotation β and θ. Specifically, the processor 5a corrects a DH (Denavit-Hartenberg) parameter of the moving device 3 based on the amounts of rotation β and @ so that the holder coordinate system Σr matches the endoscope coordinate system Ce.

The processor 5a controls the moving device 3 based on the corrected holder coordinate system Σr.

As described above, according to the present embodiment, the amounts of rotation β and θ which are manually adjusted by the operator are estimated, and the holder coordinate system Σr is corrected using the estimated amounts of rotation β and θ. Therefore, even in the case of the endoscope 2 consisting of an oblique-viewing endoscope, the operator can intuitively and accurately move the endoscope 2 in a desired direction by operating the user interface 4a while observing the endoscopic image C.

Furthermore, the amounts of rotation β and θ of the endoscope 2 can be calculated from the two endoscopic images C obtained before and after the moving device 3 is activated, respectively. In other words, it is not necessary to add any equipment such as a sensor to detect the amounts of rotation β and θ, and the amounts of rotation β and θ can be estimated without any sensor.

If a device such as a sensor for detecting the amounts of rotation β and θ of the endoscope 2 is added to the moving device 3, it would be possible to easily measure the amounts of rotation amounts β and θ. However, in this case, it is impossible to use any existing moving device 3, and it is necessary to improve the moving device 3. Moreover, the size and weight of the moving device 3 increase, and the moving device 3 may become an obstacle to the operator. Therefore, according to the present embodiment, there is no need to improve the moving device 3, and the moving device 3 can be easily miniaturized.

Further, according to the present embodiment, the area E other than the treatment tool areas D in the endoscopic image C is used for estimating the movement vector v_{obj}. As a result, even when a moving treatment tool 7 exists in the endoscopic image C, a highly accurate movement vector v_{obj} that accurately represents the moving direction of the endoscope 2 can be estimated. The amounts of rotation β and θ of the endoscope 2 can be calculated based on such a highly accurate movement vector v_{obj}, and the holder coordinate system Σr can be accurately corrected. Furthermore, based on the accurately corrected holder coordinate system Σr, the endoscope 2 can be moved by the moving device 3 in a direction that exactly corresponds to a direction input into the operating device 4 by the operator.

### (First Modification)

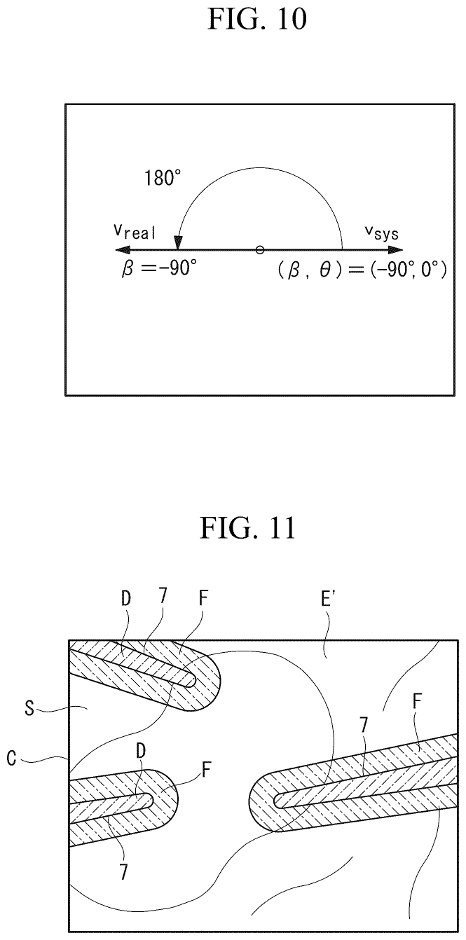

In the present embodiment, as shown in FIG. 11, a treatment tool area estimator 51 may estimate the moving direction of the object S based on another area E′ excluding areas obtained by adding margins F to the treatment tool areas D.

The margin F is an area that extends along the outline of the treatment tool area D and surrounds the treatment tool area D, and is, for example, a belt-shaped area having a predetermined width.

In the vicinity of the treatment tool 7, the movement of the object S may be affected by the movement of the treatment tool 7. For example, the object S may partially move in the vicinity of the treatment tool 7 due to the object S being pushed or pulled by the treatment tool 7.

By excluding the area obtained by adding the margin F to the treatment tool area D, it is possible to improve the estimation accuracy of the movement vectors v_{obj} and vᵣₑₐₗ Of the object S and more accurately estimate the amounts of rotation β and θ of the endoscope 2.

### (Second Modification)

Further, in the present embodiment, a plurality of temporary vanishing points Qⱼ corresponding to the amount of rotation β is set, and the vanishing point is selected using the evaluation function Uⱼ shown in Formula 2. In this case, the temporary vanishing points are set at every 30°, but they may be set at every smaller angles. As a result, the amount of rotation β can be estimated with higher accuracy.

### (Third Modification)

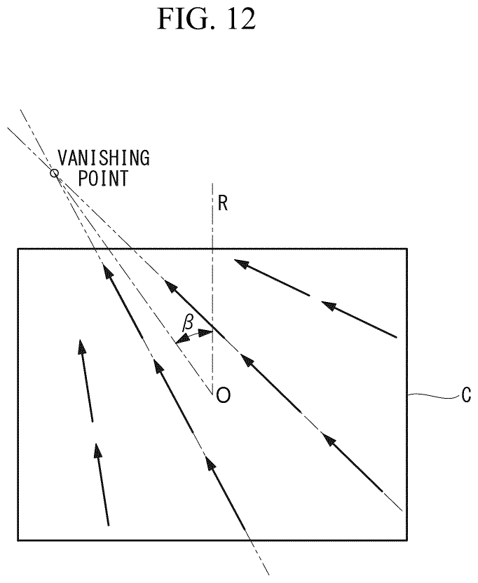

Further, instead of setting a plurality of temporary vanishing points Qⱼ corresponding to the amount of rotation β, functions of a plurality of straight lines along movement vectors v_{obj} of a plurality of feature points in the endoscopic image C are determined, and as shown in FIG. 12, the intersection of these straight lines may be calculated as the vanishing point. Then, the angle of a straight line connecting the calculated intersection point and the image center O with respect to the reference line R may be calculated as the amount of rotation β. As a result, the amount of rotation β can be estimated with higher accuracy.

### (Fourth Modification)

Further, as described above, a gap exists in the radial direction between the lens tube portion 2a of the endoscope 2 and the through-hole 8a of the trocar 8. When the moving device 3 has the passive joint 3P, there may occur a situation that even if an attempt is made to move the lens tube portion 2a in the longitudinal axis direction A, the lens tube portion 2a shifts in a direction intersecting the longitudinal axis A by the amount corresponding to the gap. The optical flow when the lens tube portion 2a is moved in the longitudinal axis direction A is used in order to estimate the amount of rotation β. Therefore, if the lens tube portion 2a shifts in the direction intersecting the longitudinal axis A, the accuracy of estimating the vanishing point and thus the amount of rotation β deteriorates.

Therefore, the lens tube portion 2a is moved in the longitudinal axis direction A sufficiently larger than the gap between the lens tube portion 2a and the trocar 8 (for example, 20 mm or more when the gap is 2 to 3 mm). As a result, it is possible to reduce the ratio of the deviation amount of the movement vector v_{obj} caused by the gap, so that the estimation accuracy of the amount of rotation β can be improved.

### (Fifth Modification)

Further, in order to accurately arrange the lens tube portion 2a of the endoscope 2 at a desired position and in a desired posture, a moving device 3 having six-degree-of-freedom may required. The moving device 3 in general as a robotic arm that can have 6 degrees of freedom, however, a moving device having 3 degrees of freedom or more can be used. In the present embodiment, as described above, the six-degree-of-freedom holder 3g holds the endoscope 2 so that the endoscope 2 is manually rotatable around the longitudinal axis A. For this reason, for example, some posture of the endoscope 2 may cause the entire endoscope 2 to rotate around the longitudinal axis A when the lens tube portion 2a of the endoscope 2 is moved in a direction perpendicular to the longitudinal axis A. In this case, the endoscopic images C obtained before and after the movement rotates, which makes it difficult to correctly calculate the moving direction of the object S.

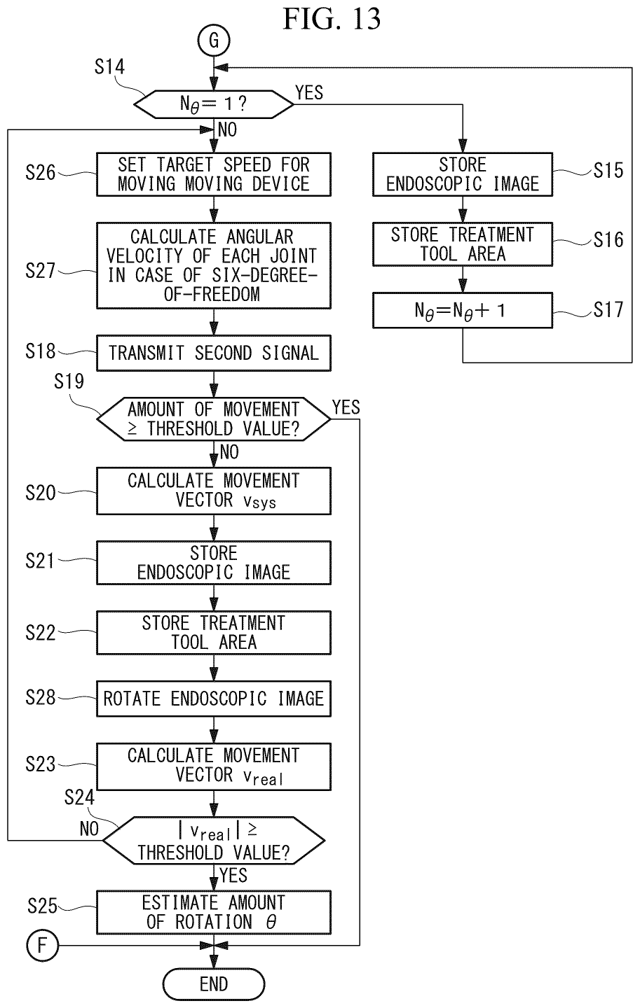

Therefore, as shown in FIG. 13, when determining in step S14 that N_{θ}=1 is satisfied, the processor 5a sets a target speed of the distal end of the moving device 3 for operating the moving device 3 (step S26). Based on the set target speed, the processor 5a calculates the angular velocity of each of the joints 3M, 3P from inverse kinematics on the assumption that the moving device 3 has six-degree-of-freedom, and calculates a second signal for achieving the calculated angular velocity (step S27).

In this case, a second signal for a joint which rotates the endoscope 2 around the longitudinal axis A, but does not actually exist is also calculated. When the thus-calculated second signal is transmitted to the moving device 3, the entire endoscope 2 would be rotated around the longitudinal axis A to compensate for the posture of the endoscope 2 if the moving device 3 has six-degree-of-freedom. However, the posture of the endoscope 2 is not compensated because the moving device 3 of the present embodiment does not have a driving joint around the longitudinal axis A.

Therefore, the processor 5a rotates the endoscopic image C stored in step S21 after the activation of the moving device 3 by only the rotation angle corresponding to the second signal calculated for the joint around the longitudinal axis A which does not actually exist (step S28). As a result, the same endoscopic image C as an endoscopic image C which would be captured when the moving device 3 has the six-degree-of-freedom can be acquired as the endoscopic image C to be captured by the endoscope 2, and at least one of the two endoscopic images C acquired before and after the transmission of the second signal is rotated by image processing so that the angles around the longitudinal axis A of the two endoscopic images C acquired before and after the transmission of the second signal match each other, whereby it is possible to accurately calculate the moving direction of the object S before and after the movement.

### (Sixth Modification)

Further, as described above, in the present embodiment, the two joints at the distal end of the moving device 3 are passive joints 3P, and have neither a motor nor an angle sensor. Therefore, when the three driving joints 3M are driven, the lens tube portion 2a of the endoscope 2 swings the trocar 8, and is moved along the through-hole 8a of the trocar 8 by the reaction force received from the trocar 8. Following this movement, the passive joints 3P are passively activated.

However, as described above, a gap exists between the inner surface of the through-hole 8a of the trocar 8 and the outer surface of the lens tube portion 2a of the endoscope 2. Therefore, as shown in FIG. 4, in a state where the lens tube portion 2a is receiving no reaction force from the trocar 8, even if the endoscope 2 is attempted to be moved in the longitudinal axis direction A by transmitting the first signal, the lens tube portion 2a may move in a direction intersecting the longitudinal axis direction A after the movement is started. In this case, since a correct movement vector v_{obj} cannot be obtained, the estimation accuracy of the amount of rotation β of the endoscope 2 deteriorates.

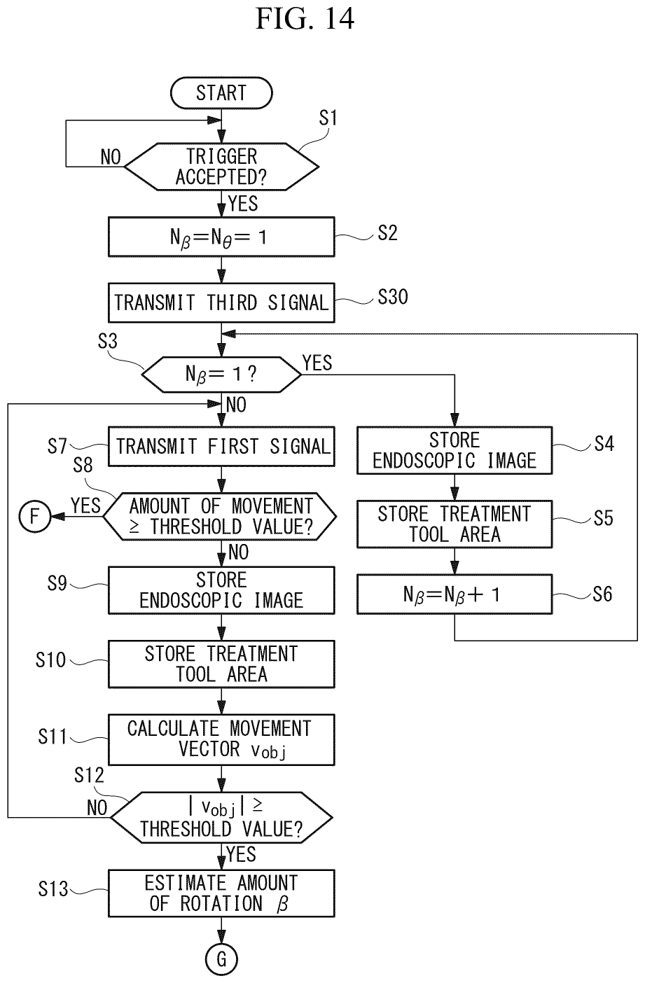

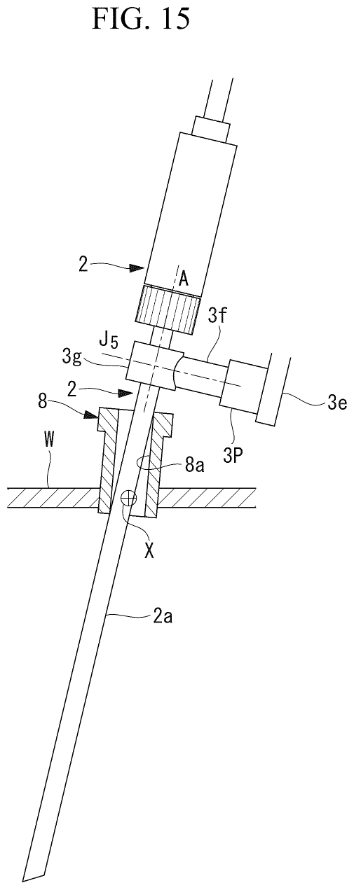

Therefore, as shown in FIG. 14, before acquiring the first endoscopic image C, the processor 5a transmits a third signal to the moving device 3 (step S30) to move the endoscope 2 to a position where the lens tube portion 2a receives a reaction force equal to or more than a predetermined threshold value from the trocar 8. For example, by transmitting the third signal, the inclination angle of the lens tube portion 2a may be changed as shown in FIG. 15 to elastically deform a sealing member (not shown) between the lens tube portion 2a and the through-hole 8a and/or the abdominal wall W, thereby generating a reaction force equal to or more than a predetermined threshold value. Thereafter, by executing the processing from step S3, it is possible to estimate the amount of rotation β with high accuracy.

### (Seventh Modification)

Further, in a case where the moving device 3 has the passive joint 3P, a problem may occur when the amount of rotation θ of the endoscope 2 is estimated. In other words, when there is a gap between the lens tube portion 2a and the through-hole 8a of the trocar 8, there may occur a situation that even if an attempt is made to move the endoscope 2 straight in a direction perpendicular to the longitudinal axis A, the passive joint 3P shifts unrestrainedly by the amount corresponding to the gap, so that the passive joint 3P cannot be moved straight.

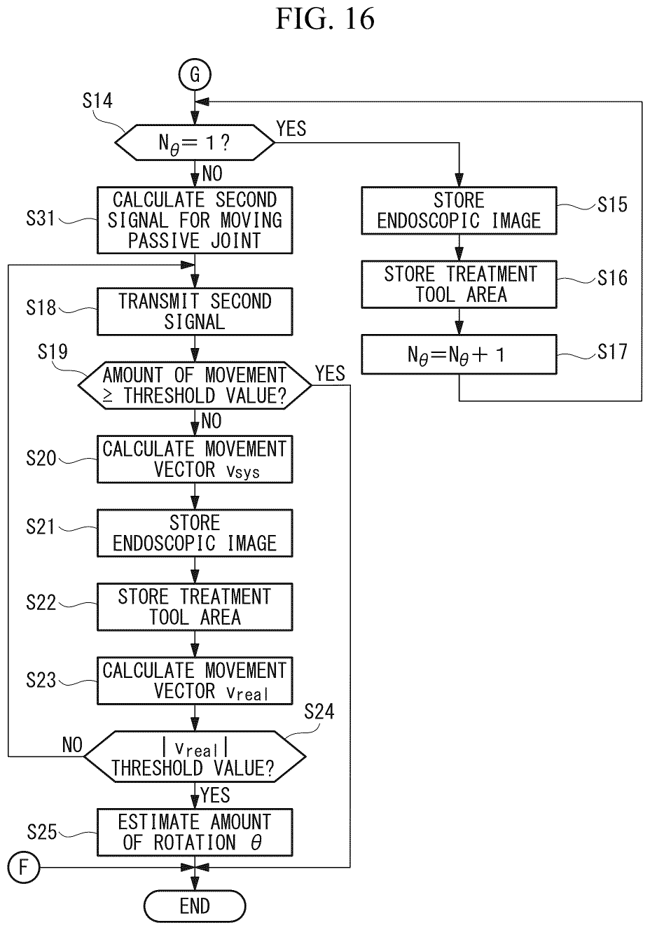

Therefore, as shown in FIG. 16, when it is determined in step S14 that N_{θ}=1 is not satisfied, the processor 5a calculates a second signal for achieving a moving direction of the endoscope 2 which allows the passive joint 3P to move in order to move the endoscope 2 straight (step S31). The processor 5a may transmit the thus-calculated second signal to the moving device 3.

Since the endoscope 2 is moved in the direction in which the passive joint 3P moves, the endoscope 2 can be moved straight in the direction perpendicular to the longitudinal axis A, and the accuracy of estimating the amount of rotation θ can be improved.

### (Eighth Modification)

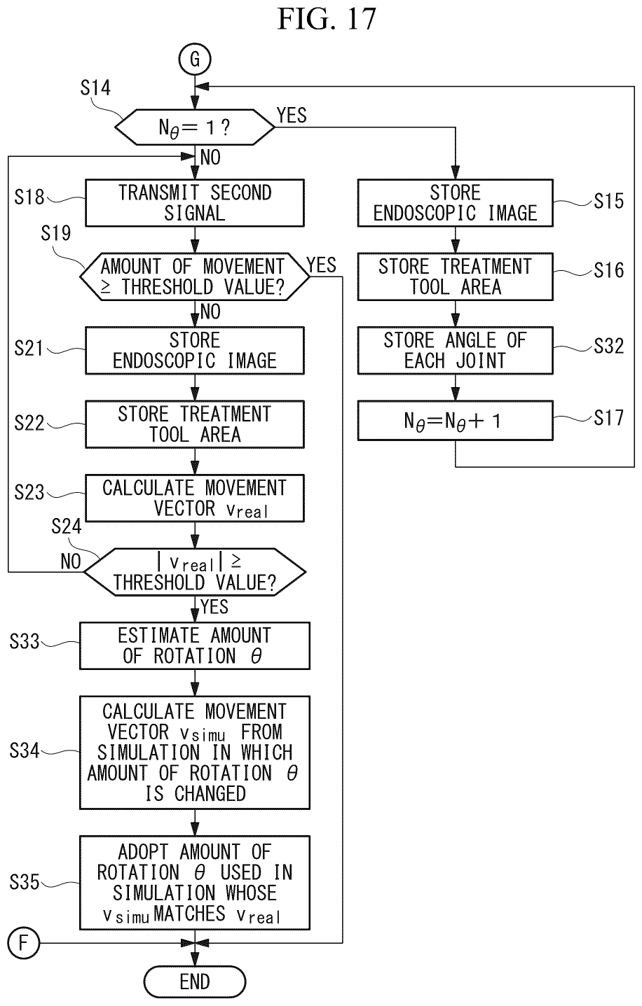

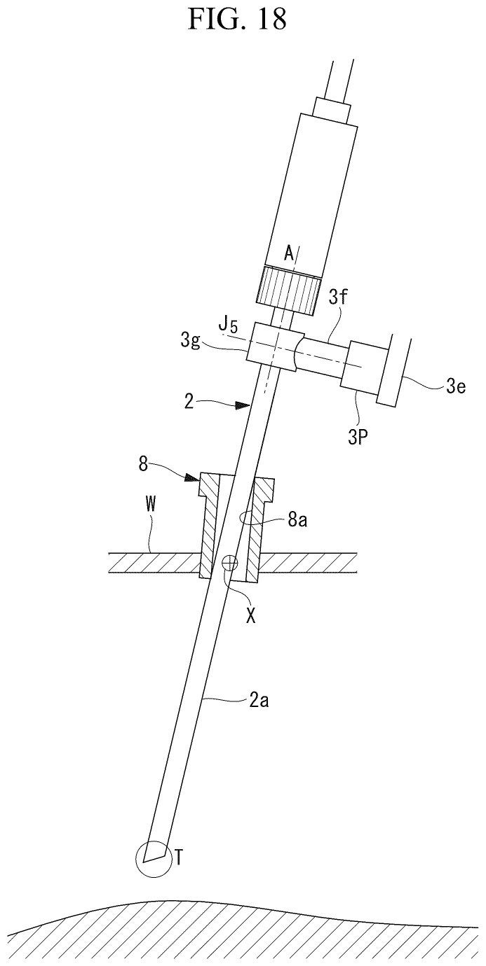

Further, in the present embodiment, in step S20, the assumed movement vector v_{sys} is calculated, and in step S25, the intersection angle between the movement vector v_{sys} and the estimated movement vector vᵣₑₐₗ is estimated as the amount of rotation θ of the endoscope 2. Instead of this, as shown in FIG. 17, the processor 5a may perform simulation for a plurality of amounts of rotation θ, calculate the movement vector of the object S on the endoscopic image C, and adopt, as an estimation result, the amount of rotation θ used in the simulation in which the moving direction matches that of the real movement vector vᵣₑₐₗ.

In other words, first, the processor 5a stores the angle of each driving joint 3M of the moving device 3 at the time when the first endoscopic image C and the treatment tool area D are stored in steps S15 and S16 (step S32). Then, at the time point when the real movement vector vᵣₑₐₗ having sufficient magnitude is calculated, the processor 5a stores the angle of each driving joint 3M of the moving device 3 again (step S33). Thereafter, the processor 5a calculates respective movement vectors vₛᵢₘᵤ of the object S through simulations in which the amount of rotation θ is changed (step S34). The processor 5a selects a simulation in which a movement vector vₛᵢₘᵤ whose moving direction matches that of the real movement vector vᵣₑₐₗ is calculated, and adopts the amount of rotation θ used in the selected simulation as an estimated value (step S35).

In the simulation, the angle of each of the joints 3M stored in steps S32 and S33, the amounts of rotation β estimated in step S13, the length of each of the links 3b, 3c, and 3d, and the distance from the distal end of the endoscope 2 to the object S are used as fixed values. Then, the processor 5a performs a plurality of simulations to calculate the movement vector vₛᵢₘᵤ of the object S using the amount of rotation θ as a parameter.

According to the present embodiment, since the simulation is performed using the real angles of the driving joints 3M of the moving device before and after movement, it is possible to calculate the movement vector vₛᵢₘᵤ in consideration of the variation in posture of the endoscope 2 caused by the moving device having five-degree-of-freedom and including the passive joints 3P. Therefore, as compared with the movement vector v_{sys} of the first embodiment which is calculated on the assumption that the distal end of the endoscope 2 moves straight in the direction perpendicular to the longitudinal axis A, the movement vector vₛᵢₘᵤ can be calculated with high accuracy, and the estimation accuracy of the amount of rotation θ can be improved.

Further, the case where the sixth to eighth modifications are implemented individually has been described, but instead of this case, the modifications may be implemented while combining at least two of these modifications.

(Ninth modification)

Furthermore, when the endoscope 2 is inserted via the trocar 8 as described above, if the endoscope 2 is excessively advanced along the longitudinal axis A in order to estimate the amount of rotation β, it is considered the distal end of the endoscope 2 comes into contact with the object S inside the abdominal cavity. On the other hand, if the endoscope 2 is excessively retreated along the longitudinal axis A, it is considered that the distal end of the endoscope 2 enters the trocar 8, and sufficient optical flow cannot be obtained.

Therefore, the operator operates the moving device 3 while checking the endoscopic image C in advance, moves the endoscope 2 to a position and posture in which the distal end thereof does not come into contact with the object S inside the body cavity, and registers the three-dimensional position of the distal end of the endoscope 2 at that time as a registration point T. The processing of estimating the amount of rotation β may be performed by moving the endoscope 2 in a retreating direction along the longitudinal axis A from a state in which the distal end of the endoscope 2 is disposed at the registration point T.

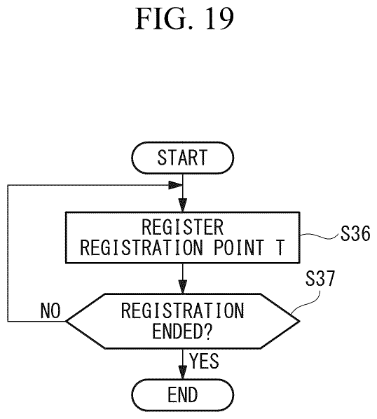

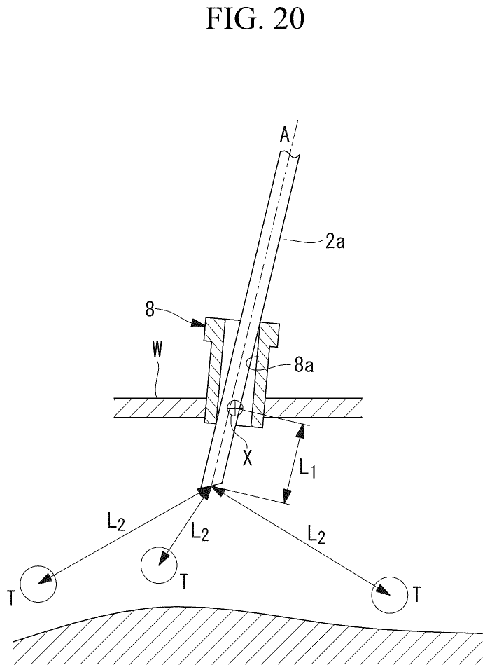

Specifically, as shown in FIG. 19, the processor 5a registers the three-dimensional position of the distal end of the endoscope 2 as a registration point T (step S36). The number of registration points T may be one or more. As shown in FIG. 20, when registering a plurality of registration points T, the processor 5a determines whether the registration is terminated (step S37), and repeats the processing of step S36 at an arbitrary number of times.

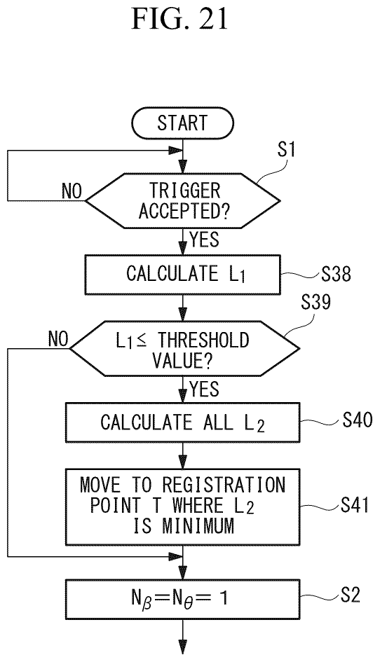

When the processing of estimating the amounts of rotation β and θ is started in a state where the distal end of the endoscope 2 is disposed at an arbitrary position, as shown in FIG. 21, the processor 5a calculates a distance L₁ between the distal end of the endoscope 2 and the pivot point X (step S38). Then, the processor 5a determines whether the distance L₁ is equal to or less than a threshold value (step S39), and if the distance L₁ is larger than the threshold value, the processor 5a executes the processing from step S2. In this case, the operating direction of the moving device 3 according to the first signal transmitted in step S7 may be either the direction in which the endoscope 2 is advanced along the longitudinal axis A or the direction in which the endoscope 2 is retreated.

When it is determined in step S39 that the distance L₁ is equal to or less than the threshold value, the processor 5a calculates a distance L₂ between the distal end of the endoscope 2 and the registration point T for all registration points T (step S40). Then, the processor 5a moves the distal end of the endoscope 2 to the registration point T which provides the smallest calculated distance L₂ (step S41), and then executes the processing from step S2. In this case, the operating direction of the moving device 3 according to the first signal transmitted in step S7 is limited to the direction in which the endoscope 2 is retreated along the longitudinal axis A.

As described above, according to the present embodiment, in the processing of estimating the amount of rotation β, it is possible to prevent occurrence of the inconvenience of entering the trocar 8 and efficiently estimate the amount of rotation β. In particular, simply by registering the registration points T, the estimation processing can be performed regardless of the position of the distal end of the endoscope 2 at the time of starting the estimation. Therefore, there is an advantage that the operator does not have to manually move the endoscope 2 to a position where the distance L1₁ is ensured to be larger than the threshold value each time.

Furthermore, when the distal end of the endoscope 2 is moved to the registration point T by the operation of the moving device 3, the processor 5a moves the distal end of the endoscope 2 as follows. First, the endoscope 2 is retreated along the longitudinal axis A until the distance L₁ between the distal end of the endoscope 2 and the pivot point X becomes a constant distance or less. Next, the posture of the endoscope 2 is adjusted so that the longitudinal axis A of the endoscope 2 is parallel to a straight line connecting a registration point T to which the endoscope 2 is requested to be moved and the pivot point X. Finally, the endoscope 2 is moved along the longitudinal axis A until the distal end of the endoscope 2 is at a distance equal to or less than a predetermined threshold value from the registration point T. As a result, even when there is an obstacle such as tissue or a treatment tool between the distal end of the endoscope 2 and the registration point T to which the endoscope 2 is requested to be moved at the start time of the processing of estimating the amount of rotation β, it is possible to move the distal end of the endoscope 2 to the registration point T without causing the distal end of the endoscope 2 to come into contact with the obstacle.

Moreover, in the present embodiment, the moving device 3 including three driving joints 3M and two passive joints 3P has been described. Instead of this moving device 3, a moving device 3 including five or more driving joints 3M may be adopted. As a result, the position and posture of the endoscope 2 can be controlled more accurately. However, since the driving joint 3M requires a motor and an angle sensor, the moving device 3 becomes larger in size. Therefore, according to the present embodiment, the minimum number of driving joints 3M that can achieve the position and posture of the endoscope 2 are provided, so that the moving device 3 can be miniaturized and the moving device 3 can be prevented from interfering with surgery.

Furthermore, in the present embodiment, the imaging element 2e in the camera head 2c is arranged so as to be orthogonal to the longitudinal axis A of the lens tube portion 2a and such that the longitudinal axis A passes through the center of the imaging surface. Instead of this, in a case where an optical axis B is inclined by the optical element 2b such as a mirror or a prism in the camera head 2c, the imaging element 2e may be arranged so as to be perpendicular to the optical axis B which is inclined with respect to the longitudinal axis A and such that the optical axis B passes through the center of the imaging surface.

### Second Embodiment

Next, an endoscope system 1, an endoscope movement control device 5, a method, a program, and a recording medium according to a second embodiment of the present disclosure will be described. In the present embodiment, points different from the first embodiment will be described, and with respect to configurations common to the first embodiment, the same reference signs are appended, and description thereof will be omitted.

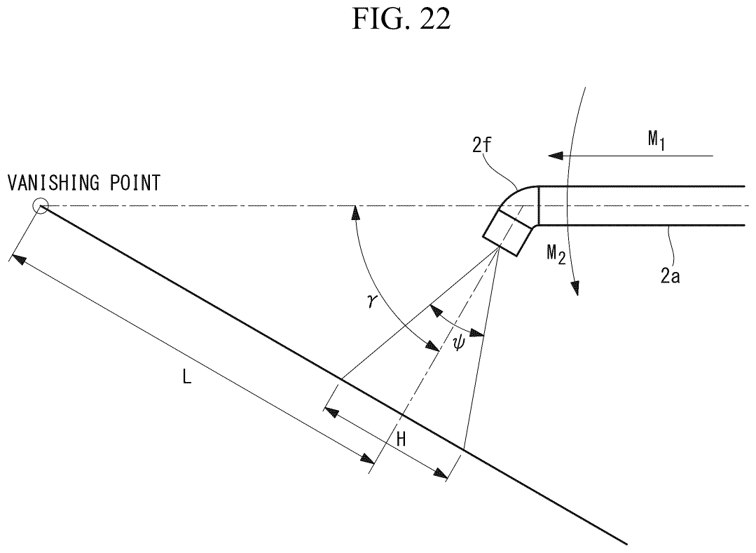

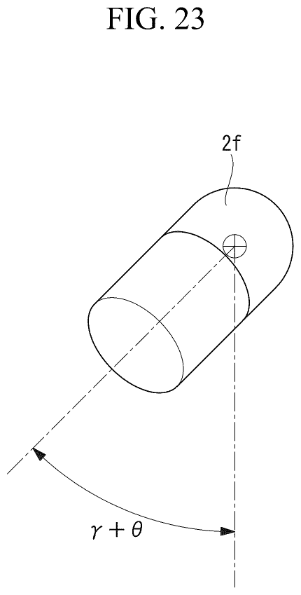

The endoscope system 1 according to the present embodiment is different from the first embodiment in that the endoscope 2 has a two-degree-of-freedom curved joint 2f at the distal end of the lens tube portion 2a as shown in FIG. 22 and FIG. 23 instead of the optical element 2b which is provided in the lens tube portion 2a to incline the visual axis B. Since the curved joint 2f has two-degree-of-freedom, there is no operation ring 2d for rotating the inclined direction of the visual axis B around the longitudinal axis A by the optical element 2b.

In the description of the present embodiment, as shown in FIG. 22, an inclination angle of the visual axis B with respect to the longitudinal axis A of the lens tube portion 2a due to the curvature of the curved joint 2f is defined as an amount of rotation γ, and as shown in FIG. 23, an angle which indicates the bending direction of the curved joint 2f with the longitudinal axis A as the center is defined as an amount of rotation δ. The two-degree-of-freedom of the curved joint 2f of the endoscope 2 is defined by these amounts of rotation γ and δ. Further, similarly to the first embodiment, the amount of rotation θ is an attachment angle of the lens tube portion 2a of the endoscope 2 to the holder 3g, and it can be arbitrarily changed manually by the operator without using the moving device 3.

In the endoscope system 1 according to the present embodiment, the processor 5a executes a program (endoscope movement control program) which is recorded in the storage unit 5c and read out to the memory 5b, thereby estimating the amounts of rotation γ, δ, and θ of the endoscope 2.

An endoscope movement control method using the endoscope system 1 according to the present embodiment will be described below with reference to the drawings.

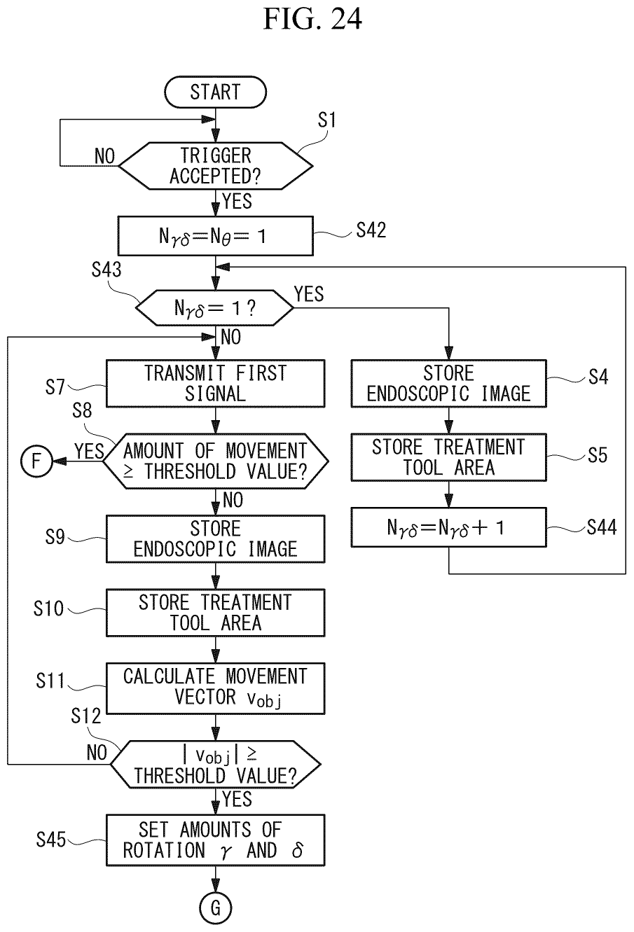

As shown in FIG. 24, the endoscope movement control method according to the present embodiment is different in that the number of loops N_{γδ} is used instead of the number of loops N_{β} in steps S42, S43, and S44, but the processing up to step S12 is performed in the same manner as in the first embodiment.

The present embodiment differs from the first embodiment in estimating the amounts of rotation γ and δ (step S45).

The amount of rotation γ can be determined by setting θ=0° and calculating Formula (3) using the distance L between the vanishing point and the image center O in the positional relation shown in FIG. 22.

$$[ Formula 3 ]$$

$$γ = {tan}^{- 1} ( \frac{2 \cdot L \cdot tan ( ψ / 2 )}{H} ) \qquad ( 3 )$$

Here, the distance L is determined as follows.

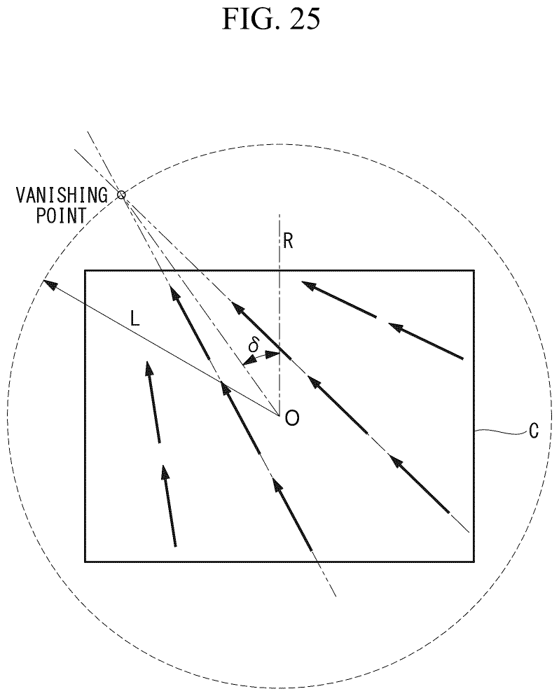

First, as shown in FIG. 25, an equation of a straight line indicating the direction of each vector is determined using movement vectors v_{obj} of two or more feature points on the endoscopic image C calculated in step S11. From the equations of these straight lines, the intersection point of the straight lines is determined as a vanishing point. As a result, the distance L from the image center O to the vanishing point is determined.

The amount of rotation γ can be estimated by substituting the determined distance L into Formula (3). Further, the angle of a straight line connecting the calculated vanishing point and the image center O with respect to the reference line R can be estimated as the amount of rotation δ. Further, the amount of rotation θ can be estimated in the same manner as in the first embodiment.

As described above, according to the present embodiment, it is possible to estimate the amounts of rotation γ and θ of the two-degree-of-freedom of the curved joint 2f of the endoscope 2 to be manually adjusted by the operator, and the attachment angle of the endoscope 2 to be manually adjusted, and correct the holder coordinate system Σr using the estimated amounts of rotation γ, δ, and θ. Therefore, even when the endoscope 2 has a curved joint 2f, the operator can intuitively and accurately move the endoscope 2 in a desired direction by operating the user interface 4a while observing the endoscopic image C.

### REFERENCE SIGNS LIST

1 Endoscope system
2 Endoscope
2a Lens tube portion (insertion portion)
2b Optical system (optical element)
2c Camera Head
2d Operation ring
2e Imaging element
3 Moving device (robotic arm)
3M Driving joint (joint)
3P Passive joint
5 Control device (endoscope movement control device)
5a Processor
8 Trocar
S Object
A Longitudinal axis
B Optical axis
M₁ First moving direction
M₂ Second moving direction
β First amount of rotation
θ Second amount of rotation

1. An endoscope system comprising:
an endoscope comprising:
an insertion portion extending in a longitudinal axis direction;
an imaging sensor disposed at a proximal end of the insertion portion so as to be rotatable about a longitudinal axis;
an optical element provided in the insertion portion to tilt an optical axis in a direction offset from the longitudinal axis direction;
a robotic arm that moves and holds the endoscope such that the endoscope is rotatable about the longitudinal axis; and
at least one processor comprising hardware, wherein the processor is configured to:
transmit, to the robotic arm, a first signal for moving the endoscope in the longitudinal axis direction,
detect a first moving direction of an object within first two images by using the first two images captured by the imaging sensor before and after the transmission of the first signal,
estimate a first amount of rotation of the optical element around the longitudinal axis with respect to the imaging sensor based on the first movement direction,
transmit, to the moving device, a second signal for moving the endoscope perpendicular to the longitudinal axis direction,
detect a second moving direction of the object within second two images by using the second two images captured by the imaging sensor before and after the transmission of the second signal, and
estimate a second amount of rotation about the longitudinal axis between the robotic arm and the endoscope based on the second moving direction and the first amount of rotation.

2. The endoscope system according to claim 1, wherein the at least one processor is configured to:
detect moving directions of a plurality of feature points of the object in the images as first moving directions,
calculate a position of an intersection point of straight lines of the plurality of feature points along the first movement directions respectively, and
estimate the first amount of rotation based on the position of the intersection point.

3. The endoscope system according to claim 1, wherein the at least one processor is configured to estimate the second amount of rotation based on a difference between the second moving direction and a direction in which the moving device moves due to the transmission of the second signal.

4. The endoscope system according to claim 1, wherein the at least one processor is configured to:
calculate a moving direction of the object in the second two images due to the transmission of the second signal by a plurality of simulation patterns using the second amount of rotation as a variable parameter, and
estimate, as the second amount of rotation, a value of the variable parameter in a simulation result which matches the second moving direction.

5. The endoscope system according to claim 1, wherein the imaging sensor includes an imaging surface disposed orthogonally to the longitudinal axis direction, and the longitudinal axis passes through an imaging center of the imaging surface.

6. The endoscope system according to claim 5, wherein the robotic arm includes a plurality of joints, and at least one of the plurality of joints is driven with a signal from the processor, to move a distal end of the insertion portion to a predetermined position in a three-dimensional space, and at the same time, control a posture of the insertion portion except for rotation about the longitudinal axis.

7. The endoscope system according to claim 6, wherein the at least one processor is configured to:
rotate at least one of the second two images captured before and after the transmission of the second signal by image processing such that angles about the longitudinal axis of the second two images captured before and after the transmission of the second signal match each other, and
detect the second moving direction.

8. The endoscope system according to claim 6, wherein
the plurality of joints includes three active joints driven with the signal, and two passive joints,
the insertion portion is inserted into a trocar, and
the processor is configured to:
transmit a third signal to the moving device to change an inclination angle in the longitudinal axis direction until the insertion portion receives a reaction force equal to or more than a predetermined threshold value from the trocar, and detect the first moving direction.

9. The endoscope system according to claim 6, wherein
the endoscope includes an operation ring fixed to the insertion portion, and
the first amount of rotation is an amount of change in a rotation angle of the operation ring about the longitudinal axis with respect to the imaging sensor.

10. The endoscope system according to claim 1, wherein the at least one processor is configured to calculate the first moving direction and the second moving direction using optical flow.

11. The endoscope system according to claim 1, wherein the robotic arm has 6-degrees of freedom.

12. An endoscope movement control method for controlling a robotic arm for moving an endoscope, the endoscope comprising an insertion portion extending in a longitudinal axis direction, an imaging sensor disposed at a proximal end of the insertion portion so as to be rotatable about a longitudinal axis, and an optical element that is provided in the insertion portion to tilt an optical axis in a direction offset from the longitudinal axis direction, and the robotic arm holding the endoscope such that the endoscope is rotatable about the longitudinal axis, the endoscope movement control method comprising:
moving the endoscope in the longitudinal axis direction by the robotic arm;
detecting a first moving direction of an object in first two images by using the first two images captured by the imaging sensor before and after the movement in the longitudinal axis direction;
estimating a first amount of rotation of the optical element about the longitudinal axis with respect to the imaging sensor based on the first moving direction;
moving the endoscope in a direction perpendicular to the longitudinal axis direction by the robotic arm;
detecting a second moving direction of the object in second two images using two images captured by the imaging sensor before and after the movement in the direction perpendicular to the longitudinal axis direction; and
estimating a second amount of rotation about the longitudinal axis between the robotic arm and the endoscope based on the second moving direction and the first amount of rotation.

13. The endoscope movement control method according to claim 12, further comprising:
detecting moving directions of a plurality of feature points of the object in the images as first moving directions, and
calculating a position of an intersection point of straight lines of the plurality of feature points along the first movement directions respectively, and
estimating the first amount of rotation based on the position of the intersection point.

14. The endoscope movement control method according to claim 12, further comprising:
estimating the second amount of rotation in consideration based on a difference between the second moving direction and a direction in which the moving device moves due to the transmission of the second signal.

15. The endoscope movement control method according to claim 12, further comprising:
calculating a moving direction of the object in the second two images due to the transmission of the second signal by a plurality of simulation patterns using the second amount of rotation as a variable parameter, and
estimating, as the second amount of rotation, a value of the variable parameter in a simulation result which matches the second moving direction.

16. The endoscope movement control method according to claim 12, further comprising:
rotating at least one of the second two images captured before and after the transmission of the second signal by image processing such that angles about the longitudinal axis of the second two images captured before and after the transmission of the second signal match each other, and
detecting the second moving direction.

17. A non-transitory computer-readable recording medium in which an endoscope movement control program for controlling a robotic arm for moving an endoscope is stored, the endoscope comprising an insertion portion extending in a longitudinal axis direction, an imaging sensor disposed at a proximal end of the insertion portion so as to be rotatable about a longitudinal axis, and an optical element that is provided in the insertion portion to incline an optical axis in a direction offset from the longitudinal axis direction, the robotic arm holding the endoscope such that the endoscope is rotatable about the longitudinal axis, and the endoscope movement control program causing a computer to:
move the endoscope in the longitudinal axis direction by the robotic arm;
detect a first moving direction of an object in first two images by using two images captured by the imaging sensor before and after the movement in the longitudinal axis direction;
estimate a first amount of rotation of the optical element about the longitudinal axis with respect to the imaging sensor based on the first moving direction;
move the endoscope in a direction perpendicular to the longitudinal axis direction by the robotic arm;
detect a second moving direction of the object in the second two images by using the second two images captured by the imaging sensor before and after the movement in the direction perpendicular to the longitudinal axis direction; and
estimate a second amount of rotation about the longitudinal axis between the robotic arm and the endoscope based on the second moving direction and the first amount of rotation.

18. The non-transitory computer-readable recording medium according to claim 17, wherein
the endoscope movement control program causing a computer to:
detect moving directions of a plurality of feature points of the object in the images as first moving directions,
calculate a position of an intersection point of straight lines of the plurality of feature points along the first movement directions respectively, and
estimate the first amount of rotation based on the position of the intersection point.

19. The non-transitory computer-readable recording medium according to claim 17, wherein
the endoscope movement control program causing a computer to:
estimate the second amount of rotation based on a difference between the second moving direction and a direction in which the moving device moves due to the transmission of the second signal.

20. The non-transitory computer-readable recording medium according to claim 17, wherein
calculate a moving direction of the object in the second two images due to the transmission of the second signal by a plurality of simulation patterns using the second amount of rotation as a variable parameter, and
estimate, as the second amount of rotation, a value of the variable parameter in a simulation result which matches the second moving direction.
